(19) United States
(12) Patent Application Publication
(10) Pub. No.: US 2013/0090618 A1
(43) Pub. Date: Apr. 11, 2013

(54) Men's Napkin

(71) Applicant: Shilash Alshammari, Khobar (SA)

(72) Inventor: Shilash Alshammari, Khobar (SA)

(21) Appl. No.: 13/613,073

(22) Filed: Sep. 13, 2012

(30) Foreign Application Priority Data
Oct. 10, 2011 (SA) 111320833.

(51) Int. Cl. A61F 13/56; A61L 15/28

(52) U.S. Cl. 604/375; 604/385.03; 604/374

(57) ABSTRACT

A men's napkin includes a triangular panel with a left, right and front portion and a fastener extending from one the right portion. The user holds the men's napkin beneath his legs with the front part facing forward and the right and left parts extending up around the left and right hips, respectively. The user then performs the following steps: 1—fold the front part (1) directly on the shaft of the penis; 2—fold the left part (3) over the front part (1); 3—fold the right part (4) over the left part (3), and 4—apply the fastener over the left part (3).

### CROSS-REFERENCE TO RELATED APPLICATIONS

This utility patent application filed under 35 USC §111 claims priority to Saudi Arabia patent application 111320833 filed on Oct. 10, 2011, which is incorporated by reference herein.

### TECHNICAL FIELD

The invention relates to the design of a napkin for personal hygiene.

### BACKGROUND

Urinary incontinence is an involuntary leakage of urine. It can be a common and distressing problem, which may have a profound impact on quality of life. Urinary incontinence almost always results from an underlying treatable medical condition but is under-reported to medical practitioners.

It is common with prostate cancer treatments. Both women and men can become incontinent from neurologic injury, congenital defects, strokes, multiple sclerosis, and physical problems associated with aging.

While urinary incontinence affects older men more often than younger men, the onset of incontinence can happen at any age. Recent estimates by the National Institutes of Health (NIH) suggest that 17 percent of men over age 60, an estimated 600,000 men, experience urinary incontinence, with this percentage increasing with age. There is also a less common but related condition for defecation known as fecal incontinence.

### SUMMARY

Formatted for easy use, and consists of one piece made of tissue used to ensure the cleanliness of the body and clothing of any wet after urinating

The invention relates to design a tissue placed on the place of the urine of man (penis) and is used to prevent wetness on the body and clothing after urination and is used for all ages, small and large and by size, and is used for patients with benign prostatic hyperplasia. It can be used for everyday use at homes, schools and hospitals.

The purpose of this invention is to maintain the body and clothes clean and free of moisture, and the patients can continue their daily lives, and reduce the necessary time to wait for the completion of urination.

In one general aspect, a men's napkin includes a triangular panel with a first, a second and a third side and a first, a second and a third vertice and a fastener extending from one or more of the first, second and third side.

Embodiments may include one or more of the following features. For example, the first side is the left side, the second side is the right side and the third side is the back side. In addition, the first vertice may be a front vertice, the second vertice may be a left vertice and the third vertice may be a right vertice.

As another feature, the fastener extends from the right side and has a top edge and a bottom edge that are parallel to an edge of the back side. The fastener may be an adhesive strip or a hook and loop fastener.

In another general aspect, a method of installing a men's napkin that includes a triangular panel with left, right and back sides with a front part at the intersection of the left and right sides and a fastener at the intersection of the right and back sides, includes positioning the men's napkin between the legs of a user with the front part facing forward, folding the front part directly onto a shaft of a penis of the user, folding the left side onto the front part, folding the right side on top of the left side, and applying the fastener on top of the left side and the front part.

### BRIEF DESCRIPTION OF THE DRAWINGS

A better understanding of the specifications and advantages of this invention can be appreciated by the illustrative example shown in the accompanying drawings and are as follows:

FIG. 1 shows illustrates features of the men's napkin.

### DETAILED DESCRIPTION

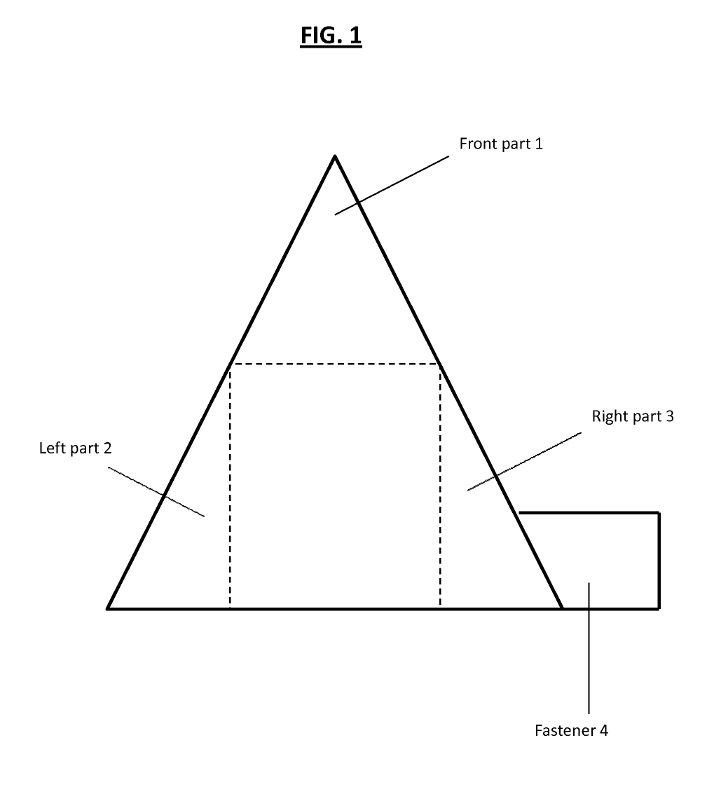

Clear from the drawings that the tissue is designed in accordance with the idea of the invention shown in FIG. (1). It consists of one piece paper napkin a triangle in shape with a fastener on the right side. The material can be made of the tissue paper or similar material to female's sanitary pads and consists of four sections: the front part (1), the right part (2), the left part (3) and a fastener (4). In FIG. 1, the area between the edges and the dashed lines show the areas highlight the front part (1), the right part (2) the left part (3).

The method of use is straightforward. The user holds the men's napkin beneath his legs with the front part facing forward such that the dashed area is under his legs and the right and left parts extending up around the left and right hips, respectively. The user then performs the following steps: 1—fold the front part (1) directly on the shaft of the penis; 2—fold the left part (3) over the front part (1); 3—fold the right part (4) over the left part (3), and 4—apply the fastener over the left part (3).

The goal is to keep the underwear clean and dry from any wetting. The invention can be made from any materials and made of different sizes as long as it is compatible with the intended use.

The elements of protection are that the napkin ensures that the body and clothing are kept clean from urine.

1. A men's napkin, comprising:
a triangular panel with a first, a second and a third side and a first, a second and a third vertice; and
a fastener extending from one or more of the first, second and third side.

2. The men's napkin of claim 1, wherein the first side comprises a left side, the second side comprises the right side and the third side comprises the back side.

3. The men's napkin of claim 2, wherein the fastener extends form the right side and comprises a top edge and a bottom edge that are parallel to an edge of the back side.

4. The men's napkin of claim 1, wherein the first vertice comprises a front vertice, the second vertice comprises a left vertice and the third vertice comprises a right vertice.

5. The men's napkin of claim 1, wherein the fastener comprises an adhesive strip.

6. The men's napkin of claim 1, wherein the fastener comprises a hook and loop fastener.

7. The men's napkin of claim 1, wherein the napkin comprises a cellulose wood fiber panel with a moisture-proof plastic shield.

8. The men's napkin of claim 1, wherein the moisture-proof plastic shield comprises polypropylene or polyethylene.

9. The men's napkin of claim 1, wherein the napkin comprises an absorbent core made up viscose fibers.

10. A method of installing a men's napkin that includes a triangular panel with left, right and back sides with a front part at the intersection of the left and right sides and a fastener at the intersection of the right and back sides, the method comprising:
positioning the men's napkin between the legs of a user with the front part facing forward;
folding the front part directly onto a shaft of a penis of the user;
folding the left side onto the front part;
folding the right side on top of the left side; and
applying the fastener on top of the left side and the front part.
